(19) United States
(12) Patent Application Publication
(10) Pub. No.: US 2022/0125706 A1
(43) Pub. Date: Apr. 28, 2022

(54) Microstructured Textile with Microencapsulated Compounds

(71) Applicant: Low Impact, LLC, Weare, NH (US)

(72) Inventors: Trenton R. Horinek, Weare, NH (US); David Horinek, Weare, NH (US)

(21) Appl. No.: 17/513,800

(22) Filed: Oct. 28, 2021

Related U.S. Application Data
(60) Provisional application No. 63/106,791, filed on Oct. 28, 2020.

(51) Int. Cl. A61K 8/96; A61B 17/54; A61K 8/02; A61K 8/11; A45D 40/00

(52) U.S. Cl. CPC A61K 8/965; A61B 17/54; A61B 2017/00761; A61K 8/11; A45D 40/00; A61K 8/0208

(57) ABSTRACT

A microstructured textile with microencapsulated compounds is used to enable a three part therapeutic delivery system. The microstructured textile can be turned into garments that passively deliver treatments to a user's skin. The microstructured textile has an a textile substrate, an abrasive material, and a microencapsulated compound. The textile substrate is an elastic material onto which the abrasive material is superimposed. The abrasive material removes dead skin when the microstructured textile is worn by the user. The microencapsulated compound is integrated into the textile substrate so that a therapeutic compound stored therein can be gradually released into the user's skin. Far infrared (FIR) emitting particles are integrated into the textile substrate. So, FIR radiation is applied to the user's skin to facilitate circulation.

The current application claims a priority to the U.S. Provisional Patent application Ser. No. 63/106,791 filed on Oct. 28, 2020.

### FIELD OF THE INVENTION

The present invention relates generally to the field of microencapsulation and textile materials. More specifically, the present invention refers to textured textile materials with mild abrasive properties that are treated with microencapsulation technology for pharmaceutical and cosmetic use.

### BACKGROUND OF THE INVENTION

According to one embodiment, a functional textile is provided comprising textured yarn coated in microencapsulation technology. The textured yarn is made of polymers embedded with volcanic minerals to provide a mild abrasive. Specifically, volcanic mud is ground into micron-sized particles and mixed into a polymer slurry. This slurry is formed into solid chips, referred to herein as masterbatch chips, then melted and combined with a base polymer and spandex to be extruded into a relatively elastic fiber. The fibers can be air-jet spun or draw textured in order to increase the fibers' overall bulk and stretch. The spun fibers are then woven to form a stretchable textile. The textile's three-dimensional stretch increases microdermabrasion and in turn, microcirculation. It is believed that an increase in circulation improves the skin's ability to absorb compounds and nutrients externally applied.

The textured textile is further functionalized through a coating of microencapsulated nutrients. The size, composition, and synthesis method of the microencapsulates depend on the nutrient formulation as well as the textile application. In a preferred embodiment, a biodegradable shell encapsulates a nutrient solution comprising Astaxanthin oil, silk amino acids (SAAs), and fulvic acid. Astaxanthin is a lipid-soluble pigment used as a dietary supplement to increase skin elasticity and lower oxidative stress due to sun damage. Silk amino acids, also known as Sericin, are water-soluble glycoproteins extracted from raw silk. Fulvic acids comprise a family of organic acids and natural compounds found in humus. The low molecular weight of Fulvic acids aid in the skin's absorption of the microencapsulated nutrients and can be also used to protect the skin from ultraviolet (UV) light damage. The textile is placed in a washer filled with the microencapsulate solution and mechanically agitated to allow the solution to penetrate to the individual fibers. The soaked textile is dried in a furnace, resulting in an enhanced textile embedded with microencapsulates throughout its fibers. Cut and sewn textiles can also be treated with the microencapsulate solution in a similar manner. The friction experienced by the enhanced textile during normal wear and use causes the microencapsulate shell to wear and eventually burst. The robustness and shear number of microencapsulates ensure the gradual release of nutrients over an extended period. Overall, Garmaceutical provides a practical and relatively long-lasting means of protecting and improving the health and appearance of the user's skin.

### BRIEF DESCRIPTION OF THE DRAWINGS

FIG. 1 is a schematic view of the present invention.

FIG. 2 is a perspective view of the textile substrate embedded with microencapsulated nutrients used in the present invention.

FIG. 3 is a perspective view of the microencapsulates gradually releasing nutrients and the nutrients being absorbed into the skin of a user of the present invention.

FIG. 4 is a schematic view of a single time-release grain used in the present invention.

FIG. 5 is a is a block diagram illustrating the three primary treatment delivery vectors provided by the present invention.

FIG. 6 is a schematic view of the manufacturing process used in the present invention.

FIG. 7 is a side view of the individual fiber containing volcanic particles.

FIG. 8 is a schematic view of the masterbatching process.

FIG. 9 is a diagram of the yarn extrusion process.

### DETAIL DESCRIPTIONS OF THE INVENTION

All illustrations of the drawings are for the purpose of describing selected versions of the present invention and are not intended to limit the scope of the present invention.

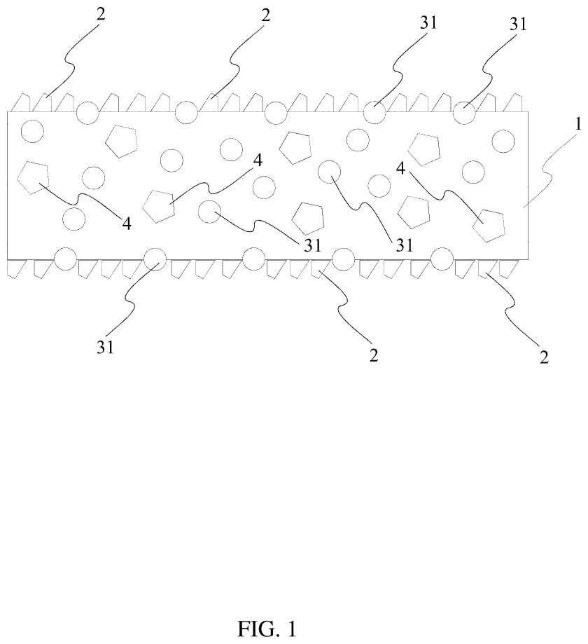

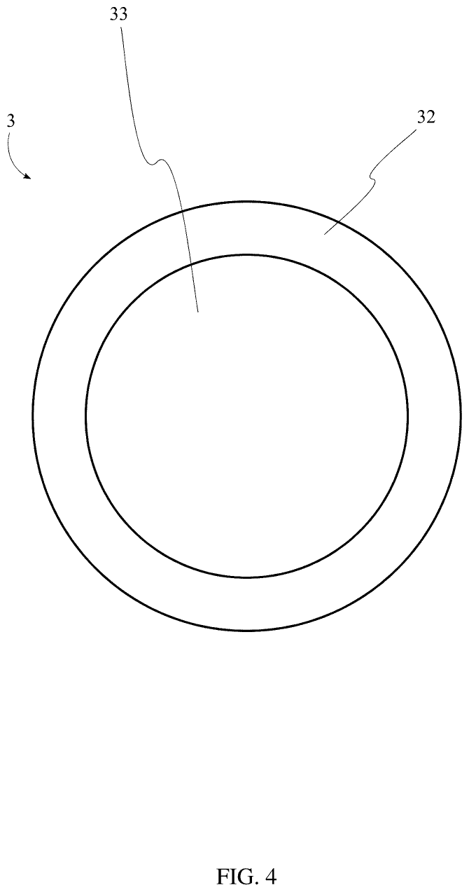

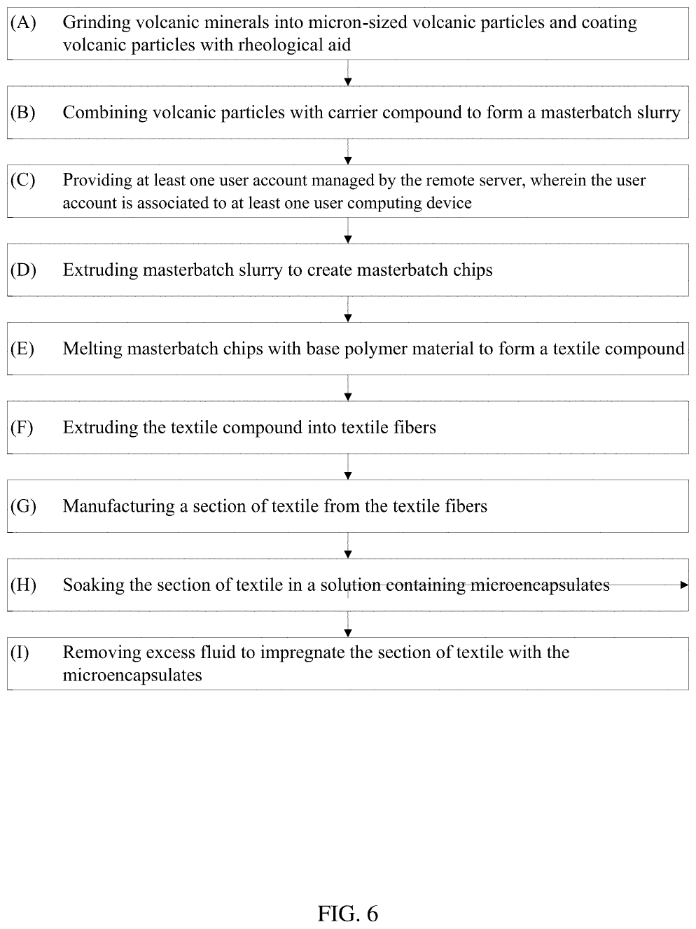

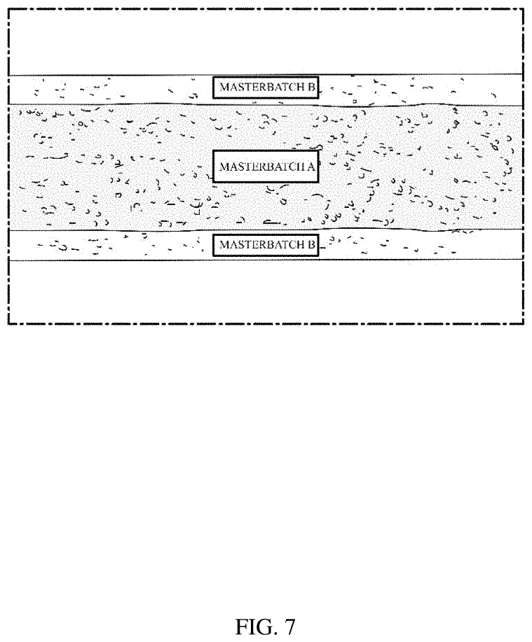

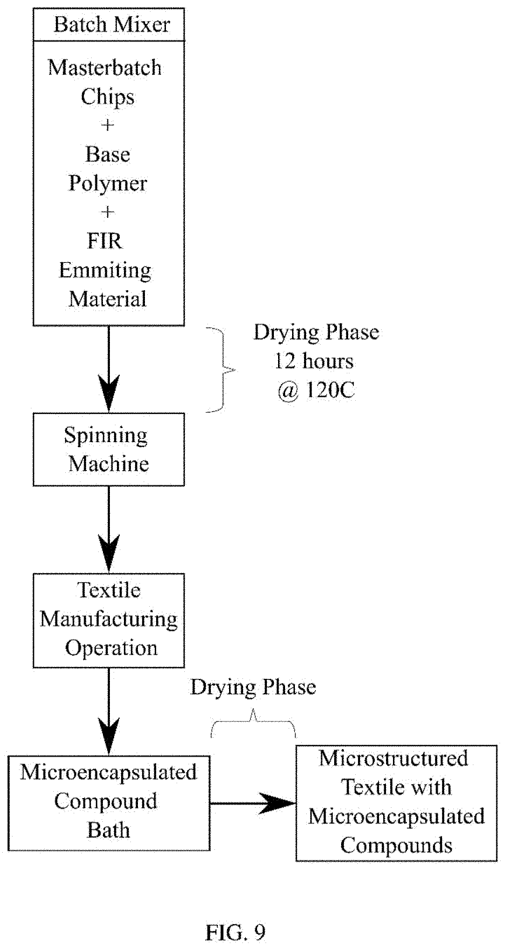

Referring to FIG. 1 through FIG. 9, the preferred embodiment of the present invention is a microstructured textile with microencapsulated compounds. The present invention provides a three-part therapeutic delivery system for treating skin conditions and improving overall health. The first aspect of this delivery system is enabled by providing an abrasive surface that performs microderm abrasion and stimulates a user's skin. The microencapsulated compound is composed of a plurality of biodegradable grains that are filled with a therapeutic additive. The microderm abrasion stimulates the skin to be more receptive to absorbing the microencapsulated compounds. The second aspect of the delivery system is provided by the microencapsulated compounds which are released over time to improve health and alleviate skin conditions. In some embodiments, a plurality of microencapsulated compounds is used to provide multiple simultaneous treatments. The third aspect of the delivery system derived from the use of far infrared (FIR) materials to generate FIR radiation when heated by the user's body. These three therapeutic delivery vectors work in concert to remove dead skin cells, administer topical care compounds, and stimulate subcutaneous structures.

Referring to FIG. 1, to achieve the above-described functionality, the present invention comprises a textile substrate 1, an abrasive material 2, and a microencapsulated compound 3. Preferably, the textile substrate 1 is a natural or synthetic fabric that serves as the structural base that is modified to provide therapeutic benefits. Specifically, the abrasive material 2 is superimposed onto the textile substrate 1. As a result, the textile substrate 1 is able to be manufactured into an abrasive garment that passively removes dead skin cells when worn. In some embodiments, the textile substrate 1 is any textile component selected from the group comprising yarn, threads, and panels of material. In further embodiments, the abrasive material 2 is mixed into the materials used to manufacture the textile substrate 1. In these embodiments, the abrasive compound is distributed within and around the textile substrate 1. The textile substrate 1 is preferably composed of an elastic material. This material choice enables the textile substrate 1 to expand and contract to perform micro abrasions that facilitate treating the user's skin without the need for the user to perform specific tasks. Preferably, the abrasive material 2 is composed of at least one volcanic mineral that is ground to micron-sized particles.

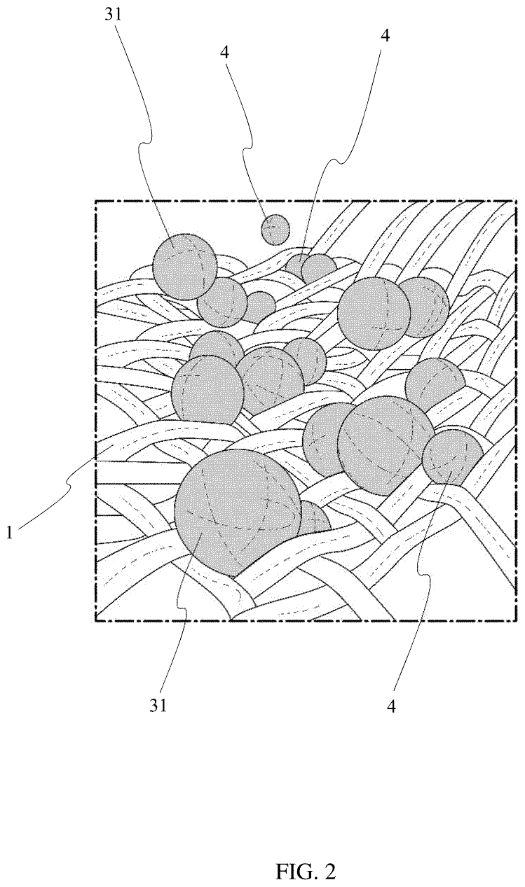

Referring to FIG. 2 through FIG. 4 the present invention is designed to provide a means of delivering therapeutic compounds to the user's skin over an extended period of time. To that end, the microencapsulated compound 3 is integrated into the textile substrate 1. Accordingly, the microencapsulated compound 3 is pressed against the user's skin while the abrasive garment is worn by the user. This configuration facilitates the gradual release of the microencapsulated compound 3 over time. Preferably, the microencapsulated compound 3 is composed of a plurality of time-release grains 31. The plurality of time-release grains 31 is composed of a collection of nutrient-filled capsules that is distributed across the textile substrate 1. As a result, the microencapsulated compound 3 is evenly delivered to the user's skin. Alternatively, the plurality of time-release grains 31 may be concentrated in a single section of the garment. Thereby, enabling targeted delivery of the therapeutic compound to specific areas of the user's body. Additionally, each of the plurality of time-release grains 31 comprises a semipermeable shell 32 and a quantity of nutrient 33. The semipermeable shell 32 is a capsule that enables the gradual release of a nutrient stored therein. The quantity of nutrient 33 is housed within the semipermeable shell 32 and the quantity of nutrient 33 is expelled through the semipermeable shell 32 over a predefined time period. As a result, the quantity of nutrient 33 can be used as an extended-release treatment. In some embodiments, the semipermeable shell 32 is composed of biodegradable materials. In supplemental embodiments, the quantity of nutrient 33 is a nutrient solution composed of a quantity of astaxanthin oil, a quantity of silk amino acids (SAAs), and a quantity of fulvic acid.

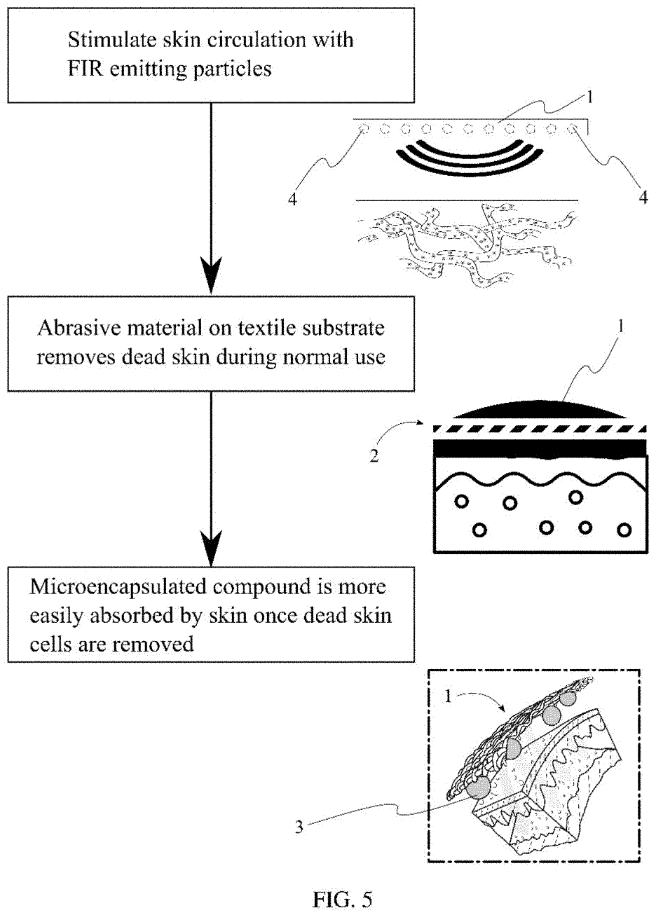

Referring to FIG. 2 and FIG. 5, the Present invention is designed to employ FIR radiation to generate therapeutic benefits. To that end, the present invention further comprises a plurality of FIR emitting particles 4. The plurality of FIR emitting particles 4 is composed of a collection of ceramic nanoparticles that are integrated into the textile substrate 1. Consequently, the plurality of FIR emitting particles 4 generate FIR radiation that is directed toward the user's body. Thereby providing therapeutic benefits including, but not limited to relaxation, better sleep, muscle recovery, joint pain relief, improved range of motion, detoxification, and improved complexion.

The present invention is an enhanced textile for microdermabrasion and skin enhancement. The system named here as textile can include, but is not limited to, fibers, yarns, weave, mat, or cloth having a general two-dimensional structure, i.e., a width and a length which are significantly larger than a thickness. The enhanced textiles can be made through a mechanical process of weaving and the like or be non-woven, whereby a plurality of textured fibers are bonded, interlocked, or otherwise joined. Further, the enhanced textile can be used to manufacture different garments, wearable devices, and products where non-enhanced textiles are traditionally used.

Referring now to FIG. 1-9, in one embodiment, the enhanced textile is made through a masterbatching and extrusion process. The masterbatching process consists of extruding and cutting a polymer-based slurry into a plurality of masterbatch chips, as shown in FIG. 2. The slurry, referred to herein as a masterbatch, is composed of a volcanic mix and a carrier. The volcanic mix is composed of a plurality of volcanic particles and at least one rheological aid. The volcanic particles are made by drying volcanic mud, grinding the mud, then sorting the mud particles based on size and hardness. In the preferred embodiment, volcanic particles with a particle size of d₁₀₀ of 1 micron, i.e., a 100% particle size distribution less than 1 micron, and mean diameter of 0.5 microns are utilized. Ideally, the particles should not be too hard so as to provide a mild abrasive texture in the final enhanced textile. In the preferred embodiment, the volcanic particles should have a hardness of six to seven on the Mohs hardness scale. Furthermore, the volcanic particles may contain the following elements: B, Na, Mg, Al, Si, K, Ca, Sc, Ti, V, Cr, Mn, Fe, Co, Ni, Cu, Zn, Ga, Ge, Se, Rb, Sr, Zr, Nb, Ru, Ag, In, Sn, Sb, Ba, Ce, Ir. It is thought that the combination of these elements provides the enhanced textile an improved ability to harness energy from the user's body heat.

The ground and sorted volcanic particles are coated in a rheological aid in order to prevent agglomeration during the masterbatching process. Possible rheological aids include a wax or similar low-density oxidized polyethylene. A 1 to 10 weight percent (wt %) of rheological aid to volcanic particles can be utilized. In the preferred embodiment, a 3 wt % of rheological aid is used to coat the volcanic particles. 5 to 20 wt % of volcanic mix is added to the carrier. Suitable carriers include PET, PP, PLA, PBA, RPET, Nylon, Nylon 66, synthetic silk, Acrylic, Olefin, Modacrylic, Spandex, Aramids, rayon, Lyocell, and other synthetic or natural carriers. As shown in FIG. 2, the masterbatch can be extruded into a plurality of masterbatch chips by means of a twin screw extruder. The extruded masterbatch is then cut into the desired size and allowed to solidify. Other masterbatch chip manufacturing methods may also be utilized.

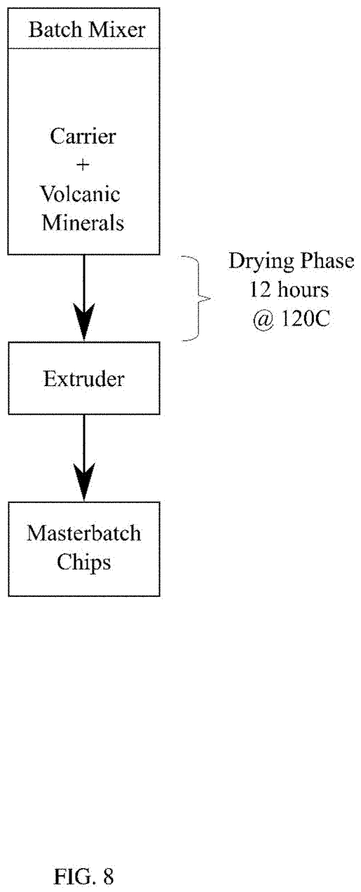

Referring now to FIG. 8, in an embodiment the enhanced textile comprises at least one textured yarn. The textured yarn may be monofilament or multifilament and comprises at least one textured fiber. The textured fiber is made through the extrusion of the plurality of masterbatch chips. The solid masterbatch chips are heated and combined with at least one base polymer. Possible base polymers such as PET, PP, PLA, PBA, RPET, Nylon, Nylon 66, synthetic silk, Acrylic, Olefin, Modacrylic, Spandex, Aramids, rayon, Lyocell, and other synthetic or natural polymers may be utilized. In addition, spandex may be added to improve the enhanced textile's elasticity. In the preferred embodiment, at least 3 wt/o spandex is incorporated to provide a pliable textile. The masterbatch chips are melted and combined with the at least one base polymer and spandex then extruded into a textured fiber. The textured fibers can be spun to create a monofilament or multifilament textured yarn. The textured yarn can be further spun to have a S-twist, Z-twist, or a combination thereof, as well as other textures. Possible texturizing methods include air-jet spinning and draw texturing, but other methods can be used as known in the art. The textured yarn can be formed into a textured textile of various constructions using methods such as 3D knitting, warp knitting, circular knitting, seamless knitting, and weaving.

To create the enhanced textile, the textured textile is soaked in a compound solution. The compound solution is composed of a carrier solution and a plurality of microencapsulates. The carrier solution is composed of a carrier oil, silk amino acids, Astaxanthin oil, fulvic acid, and a pH balancer. In the preferred embodiment, a 1 wt % of Astaxanthin oil to carrier solution is added to ethanol in a 1:20 weight ratio of Astaxanthin oil to ethanol then sprayed onto the silk amino acids. The SAAs are dried to form a thin coating of Astaxanthin. The Astaxanthin-coated SAAs can provide sun protection by down converting UV light into red light, i.e., wavelengths around 660 nanometers. 2 wt % of fulvic acid to carrier solution is added to aid in nutrient absorption as well as to protect the skin from UV damage and promote collagen growth for more youthful-looking skin. In the preferred embodiment, grape seed oil is used as the carrier oil and citric acid is used as the pH balancer to achieve a pH of 4.6 for the overall carrier solution.

A variety of microencapsulation methods may be utilized to encapsulate the carrier solution. For example, coacervation, droplet gelation, solvent evaporation, polymerization, gelation, as well as other microencapsulate techniques known in the art may be used. In the preferred embodiment, the microencapsulates are 10 to 15 microns in diameter. The microencapsulate walls are made from edible gums, resins, or other suitable biodegradable materials. The plurality of microencapsulates and leftover carrier solution from the microencapsulation process form the compound solution which is used for the textile bath. The textile bath process consists of placing the textured textile into a washer with a diluted compound solution, herein referred to as a bath solution. The bath solution is a 50:1 weight ratio of water to compound solution which coats the textured textile with a plurality of microencapsulates. After soaking and mechanically agitating the enhanced textile within the bath solution, the bath solution is drained and can be utilized for future baths. The coated enhanced textile is placed in a dryer at around 100 to 200 degrees Celsius. In one embodiment, the coated enhanced textile is dried at 160 degrees Celsius. The relatively high drying temperature causes the plurality of microencapsulates to bond to the enhanced textile. Textured textiles that have been cut and sewn can also be washed in the bath solution.

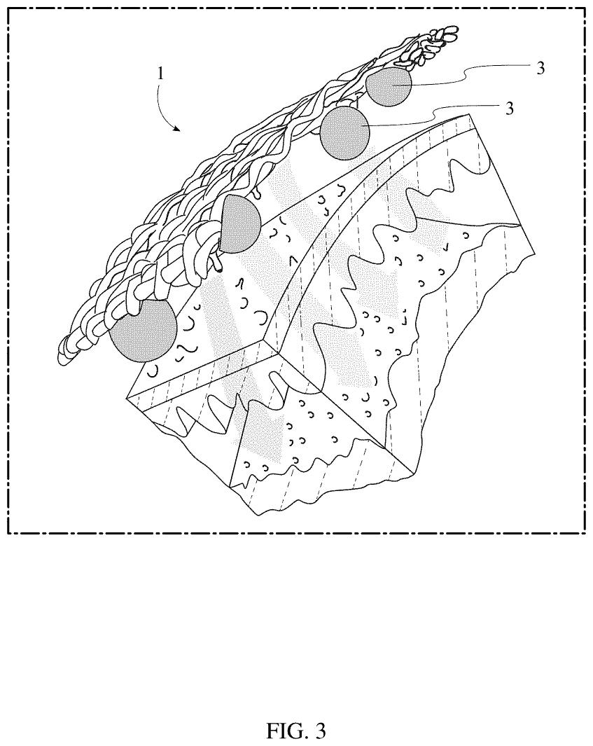

Referring now to FIG. 2-3, the enhanced textile is depicted comprising the enhanced textile embedded with microencapsulated nutrients. The mechanical wear and exposure to environmental factors cause the microencapsulate walls to thin. Eventually, the microencapsulate walls burst and release the microencapsulated nutrients. The improved microcirculation provided by the mildly abrasive volcanic particles and the use of low density fulvic acid together enhance the nutrients' absorption by the skin. Furthermore, the robustness of the microencapsulate walls and shear number of microencapsulates ensure a steady and prolonged release of nutrients for long term pharmaceutical and cosmetic treatments.

Although the invention has been explained in relation to its preferred embodiment, it is to be understood that many other possible modifications and variations can be made without departing from the spirit and scope of the invention as hereinafter claimed.

What is claimed is:

1. A microstructured textile with microencapsulated compounds comprising:
a textile substrate;
an abrasive material
a microencapsulated compound;
the abrasive material being superimposed onto the textile substrate; and
the microencapsulated compound being integrated into the textile substrate.

2. A microstructured textile with microencapsulated compounds as claimed in claim 1, wherein the textile substrate being composed of an elastic material.

3. A microstructured textile with microencapsulated compounds as claimed in claim 1, wherein the abrasive material being composed of volcanic minerals.

4. A microstructured textile with microencapsulated compounds as claimed in claim 1 comprising:
the microencapsulated compound comprising a plurality of time-release grains; and a quantity of nutrient; and
the plurality of time-release grains being distributed across the textile substrate.

5. A microstructured textile with microencapsulated compounds as claimed in claim 4 comprising:
each of the plurality of time-release grains comprising a semipermeable shell and a quantity of nutrient;
the quantity of nutrient being housed within the semipermeable shell; and
the quantity of material being expelled through the semipermeable shell over a predefined time period.

6. A microstructured textile with microencapsulated compounds as claimed in claim 1, wherein the semipermeable shell being composed of biodegradable materials.

7. A microstructured textile with microencapsulated compounds as claimed in claim 1, wherein the quantity of nutrient being a nutrient solution comprising a quantity of astaxanthin oil, a quantity of silk amino acids (SAAs), and a quantity of fulvic acid.

8. A microstructured textile with microencapsulated compounds as claimed in claim 1 comprising:
a plurality of far infrared (FIR) emitting particles; and
the plurality of FIR emitting particles being integrated into the textile substrate.

9. A method of manufacturing a microstructured textile with microencapsulated compounds comprising:
(A) grinding volcanic minerals into micron-sized volcanic particles and coating volcanic particles with rheological aid;
(B) combining volcanic particles with carrier compound to form a masterbatch slurry;
(C) extruding masterbatch slurry to create masterbatch chips
(D) melting masterbatch chips with base polymer material to form a textile compound;
(E) extruding the textile compound into textile fibers;
(F) generating a section of textile from the textile fibers;
(G) soaking the section of textile in a solution containing microencapsulates; and
(H) removing excess fluid to impregnate the section of textile with the microencapsulates.
